(19) United States
(12) Patent Application Publication
(10) Pub. No.: US 2010/0310670 A1
(43) Pub. Date: Dec. 9, 2010

(54) SUSTAINED-RELEASE MICROSPHERE CONTAINING SHORT CHAIN DEOXYRIBONUCLEIC ACID OR SHORT CHAIN RIBONUCLEIC ACID AND METHOD OF PRODUCING THE SAME

(75) Inventors: Hiroaki Okada, Tokyo (JP); Yuki Takashima, Tokyo (JP); Naoyuki Murata, Tokyo (JP)

Correspondence Address: FOLEY AND LARDNER LLP;SUITE 500, 3000 K STREET NW, WASHINGTON, DC, 20007, US

(73) Assignee: TAKEDA PHARMACEUTICAL COMPANY LIMITED, Tokyo (JP)

(21) Appl. No.: 12/065,255

(22) PCT Filed: Mar. 3, 2006
(86) PCT No.: PCT/JP2006/304089

§ 371 Date: Sep. 2, 2009

(30) Foreign Application Priority Data
Sep. 2, 2005 (JP) 2005-254966

(51) Int. Cl. A61K 31/7105; C07H 21/04; C07H 21/02; A61K 31/711; A61P 29/00; A61P 31/12; A61K 9/14

(52) U.S. Cl. 424/501; 536/23.1; 536/24.5; 514/44 R; 525/54.2

(57) ABSTRACT

A sustained-release microsphere formulation containing a short chain deoxyribonucleic acid or a short chain ribonucleic acid as an active ingredient, which has improved sustained-release properties and long-lasting efficacy, is provided. A fine particle formulation, encapsulating stably a short chain deoxyribonucleic acid or a short chain ribonucleic acid, being capable of inhibiting, for a long period, expression of a specific protein related to a disease, and which can be administered by injection or transmucosally, and a production method of the same are provided. A sustained-release microsphere formulation containing a short chain deoxyribonucleic acid or a short chain ribonucleic acid, particularly siRNA, as an active ingredient, especially a sustained-release microsphere prepared through a w₁/o/w₂ type emulsion, is characterized in that a positively charged basic substance, such as arginine, polyethylenimine, a cell permeable peptide, poly-L-lysine or poly-L-ornithine, is included in an in vivo degradable polymer.

### TECHNICAL FIELD

The present invention relates to a sustained-release microsphere, in which a short chain ribonucleic acid (siRNA; small interfering RNA) inhibiting the expression of a specific protein, especially a disease related protein, is enclosed by an in vivo degradable polymer, and a required dose of the siRNA is released stably and persistently for a long period, and to a method of producing the same. The sustained-release microsphere is especially useful for injections, and also usable for administration to nasal, bronchial or pulmonary mucosa.

### BACKGROUND ART

The base sequence of the human genome has been recently decoded and all the human genetic information has been revealed. Thereafter studies on functional genomics are carried out energetically and more details on human genes are being clarified. As a result, cell signal transduction mechanisms, cell proliferation and differentiation mechanisms, etc. have been made clear, and influence on body functions by promotion and inhibition of protein expression, or relationship between various genetic abnormalities and diseases have been clarified, and studies for applying human genes to medical treatments are being actively continued.

Among others, a so-called antisense technique is known, by which technique a strand with a sequence to pair with a specific gene related to a disease inhibits the expression of the gene. In practice, an oligo-RNA or an oligo-DNA is synthesized, and recently their derivatives or RNA/DNA-chimera molecules have been designed. The highest hurdle to the realization of an antisense medicine is a way of delivering the medicine to a cell.

Recently, an antisense therapy using a short single stranded antisense DNA or RNA, and an siRNA (small interfering RNA) technique, whereby mRNA is degraded sequence-specifically in a cell by RNAi (RNA interference) with a short double stranded RNA (dsRNA) to inhibit the expression of a specific gene, have been attracting broad attention as a new pharmacotherapy of intractable diseases.

Especially the siRNA technique has drawn a strong interest, because a smaller dose can be effective compared with a conventional antisense therapy. Reportedly, siRNA with 21 to 29 base pairs (bps) can effectively knock down a target gene.

JP Patent Publication (Kokai) No. 2005-192556 A (2005) reports a long dsRNA for RNAi (double stranded RNA for interference), by which gene expression is inhibited effectively regardless of a target site, in which the cytotoxicity is low, and an interferon response is mitigated. (Patent Literature 1)

JP Patent Publication (Kohyo) No. 2005-508306 A (2005) reports a method for inhibiting the expression of a mammal gene by RNAi, and the use of the relevant composition for academic and therapeutic field. (Patent Literature 2)

JP Patent Publication (Kokai) No. 2005-73573 A (2005) reports a method for suppressing the production of prion protein, which is a causing factor of an intractable disease of BSE, and application of an RNAi technique to the method. (Patent Literature 3)

JP Patent Publication (Kohyo) No. 2004-535813 A (2004) reports a method of selective post-transcriptional gene silencing of expression of an exogenous gene originated from virus using siRNA in a mammal cell. (Patent Literature 4)

Similar to intake or administration of an ordinary medicine, also for a gene-based medicine, a so-called drug delivery system (DDS), by which a gene of interest is introduced surely into a target body site or specifically to a specific target tissue, is utilized to suppress a side effect and to enhance the therapeutical efficacy of a gene medicine. However the drug delivery system does not work well for single use of a gene, and a combination with a gene carrier has been tried. For example, a receptor on the cell surface is selected as a target, and a gene carrier is modified with a ligand of the receptor. For example, J. Control Release, 74, 341 (2001) reports a case, wherein a gene carrier is modified with VEGF. (Non-Patent Literature 1)

J Drug Target, 12, 393-404 (2004) reports a preparation of sustained-release particles, wherein antisense-oligonucleotide, ribozyme, ribonucleic acid, such as siRNA, or oligonucleotide bonded with a lipophilic substance, such as cholesterol, is encapsulated in an in vivo degradable polymer of poly-lactic acid/glycolic acid, and reports further the release properties of the sustained-release particles and the effect of the in vivo gene expression inhibition. (Non-Patent Literature 2)

However there remain problems for practical use of the gene medicines, such that, due to extremely high polarity of a short chain ribonucleic acid and ribonucleic acid, their biomembrane permeability is limited, and due to extremely fast metabolism after administration into the body by enzymes in the body, the administration into gastrointestinal tract or blood is not very effective, and in case of local application the efficacy does not last.

The sustained-release formulation technology has been using a method for producing a single composition microsphere using an appropriate single liquid preparation mixture of a biodegradable polymer, a drug, an additive and a solvent, by spray drying or other production processes, in order to produce a formulation form enabling delivery of drug little by little at a constant rate. As a method for producing a microsphere formulation, a method for producing sustained-release microspheres from a w/o/w emulsion is well known, which emulsion is prepared by forming a w/o emulsion by adding aqueous solution of a bioactive peptide, etc. as an internal aqueous phase to an solution of an in vivo degradable polymer in an organic solvent as an oil phase, and adding the emulsion into water. To obtain the optimum pharmacological effect of a sustained-release formulation in vivo for a definite period, the initial release amount and the release rate during the following release period of a drug should be appropriately regulated. The initial release amount and the release rate have been regulated so far by changing the above-indicated parameters for the production of microspheres, such as the type and concentration of a biodegradable polymer, the content of a drug, the quantity of an additive controlling the release rate, and the quantity of a solvent.

For a sustained-release formulation, as general production methods of a sustained-release drug delivery system (DDS) formulation, capsulation by a coacervation method, an emulsion phase separation method, or a spray drying method, and a solvent evaporation method in an organic or aqueous phase are known. Among those methods, a solvent evaporation method in an aqueous phase is most frequently used, which is roughly classified into an emulsion (w/o/w; water/oil/water) evaporation method and a single emulsion (o/w; oil/water) evaporation method.

The w/o/w method, which is mainly used for encapsulation of a water soluble drug, such as a peptide or a protein, is a method dispersing an aqueous solution containing a drug produced by dissolving the drug into an aqueous solution, into an organic solvent containing a biodegradable polymer to form a primary emulsion (water in oil), and then dispersing the same into an aqueous phase. The o/w method, which is mainly used for encapsulation of a lipophilic drug, is a method dissolving both a drug and a biodegradable polymer in an organic solvent or a mixture of organic solvents (oil), and dispersing the same into an aqueous phase. In both the methods, a polymer in an organic solvent phase solidifies to form microspheres due to decrease in the polymer's solubility caused by removal of an organic solvent by extraction or evaporation in the course of being dispersed into an aqueous phase. Generally, the microspheres produced by the w/o/w method are more porous than those produced by the o/w method, and therefore are characterized in that the surface area is larger to give a relatively higher initial release rate of a drug.

(Patent Literature 1): JP Patent Publication (Kokai) No. 2005-192556 A (2005)

(Patent Literature 2): JP Patent Publication (Kohyo) No. 2005-508306 A (2005)

(Patent Literature 3): JP Patent Publication (Kokai) No. 2005-73573 A (2005)

(Patent Literature 4): JP Patent Publication (Kohyo) No. 2004-535813 A (2004)

(Non-Patent Literature 1): E. K. Gaidamakova. J. Control Release, 74, 341 (2001)

(Non-Patent Literature 2): Alim Khan, Mustapha Beenboubetra. J Drug Target, 12, 393-404 (2004)

### DISCLOSURE OF THE INVENTION

An object of the present invention is to provide a sustained-release microsphere, which stably encapsulates a short chain deoxyribonucleic acid or a short chain ribonucleic acid, and is able to inhibit, for a long period, expression of a specific protein, especially a protein related to a disease, especially a sustained-release microsphere containing a basic substance which can form a complex with the nucleic acid, and a production method thereof.

Generally, if a pharmaceutical formulation containing a nucleic acid, a peptide and a protein is administered orally or parenterally, it is degraded by enzymes in the body, and the efficacy of the pharmaceutical formulation disappears quickly. Various trials have been made to conquer the problem. One of which is to formulate a long sustained-release injectable.

The present inventors studied intensively for achieving the object to solve the above object and discovered that a positively charged basic substance makes a microsphere, especially a sustained-release microsphere prepared through a w₁/o/w₂ type emulsion, encapsulate at a high inclusion rate a short chain deoxyribonucleic acid or a short chain ribonucleic acid, thereby completing the present invention.

Namely, the present invention provides a sustained-release microsphere formulation made with an in vivo degradable polymer containing a short chain deoxyribonucleic acid or a short chain ribonucleic acid, and a positively charged basic substance.

According to the present invention, in order to deliver stably and persistently a short chain deoxyribonucleic acid and a short chain ribonucleic acid to a target cell, the object sustained-release microsphere can be produced by encapsulation in a so-called in vivo degradable polymer, which has biodegradability and biocompatibility, of a short chain deoxyribonucleic acid or a short chain ribonucleic acid by a microcapsule production method, such as a w₁/o/w₂ emulsion drying-in-liquid technique.

Further detail will be described below.

1. A sustained-release microsphere comprising a short chain deoxyribonucleic acid or a short chain ribonucleic acid as an active ingredient and 1 weight % to 10 weight % of a positively charged basic substance which can form a complex with the nucleic acid by means of electrostatic interaction.

2. The sustained-release microsphere according to 1 hereinabove, wherein the short chain deoxyribonucleic acid or the short chain ribonucleic acid has a single strand or double strand structure, and the length of 15 to 85 bases.

3. The sustained-release microsphere according to 1 hereinabove, wherein the short chain deoxyribonucleic acid or the short chain ribonucleic acid has a single strand or double strand structure, and the length of 15 to 30 bases.

4. The sustained-release microsphere according to any one of 1 to 3 hereinabove, wherein the short chain ribonucleic acid is siRNA with the length of 15 to 30 bases.

5. The sustained-release microsphere according to any one of 1 to 4 hereinabove, wherein the positively charged basic substance is a cationic polymer.

6. The sustained-release micro sphere according to any one of 1 to 4 hereinabove, wherein the positively charged basic substance is selected from the group consisting of arginine, polyethylenimine (PEI), a cell permeable peptide, poly-L-lysine, poly-L-ornithine, and siLentFect®.

7. The sustained-release microsphere according to 6 hereinabove, wherein the positively charged basic substance is selected from the group consisting of polyethylenimine (PEI), a cell permeable peptide, poly-L-lysine, poly-L-ornithine, and siLentFect®.

8. The sustained-release microsphere according to any one of 1 to 7 hereinabove, which further comprises an in vivo degradable polymer.

9. The sustained-release microsphere according to 8 hereinabove, wherein the in vivo degradable polymer is a copolymer of polylactic acid and polyglycolic acid or a copolymer of lactic acid and glycolic acid.

10. The sustained-release microsphere according to any one of 1 to 9 hereinabove, wherein the short chain deoxyribonucleic acid or the short chain ribonucleic acid as an active ingredient can be injected intradermally, subcutaneously, or intramuscularly, into an eyeball, a joint, an organ tissue, a tumor tissue.

11. A pharmaceutical composition comprising the sustained-release microsphere according to any one of 1 to 10 hereinabove as an active ingredient.

12. An anticancer agent comprising the sustained-release microsphere according to any one of 1 to 10 hereinabove as an active ingredient, wherein the short chain deoxyribonucleic acid or the short chain ribonucleic acid can inhibit growth of tumor cells.

13. A method, based on a w₁/o/w₂ emulsion drying-in-liquid technique, for producing the sustained-release microsphere according to any one of 1 to 10 hereinabove, characterized in that the method comprises the steps of:

forming a w₁/o emulsion by mixing with high speed agitation an internal aqueous phase prepared by dissolving siRNA in the presence of a positively charged basic substance, into an oil phase prepared by dissolving an in vivo degradable polymer in an organic solvent;

forming a w₁/o/w₂ emulsion by adding the w₁/o emulsion into an external aqueous phase solution with agitation; and

drying the same.

14. A method for producing the sustained-release microsphere according to any one of 1 to 10 hereinabove, characterized in that a w/o, o/w or s/o emulsion through a w₁/o/w₂ or s/o/w emulsion, is subjected to solvent removal in a supercritical fluid or spray drying.

15. The production method according to 14 hereinabove, characterized in that an organic solvent having compatibility with a continuous oil phase, but not solubility of an in vivo degradable polymer, is gradually added to an external oil phase through a w/o emulsion or an s/o suspension to have the short chain deoxyribonucleic acid or the short chain ribonucleic acid encapsulated.

16. The production method according to 15 hereinabove, wherein the in vivo degradable polymer is a copolymer of polylactic acid and polyglycolic acid or a copolymer of lactic acid and glycolic acid.

According to the present invention, by the use of a positively charged substance, a short chain deoxyribonucleic acid or a short chain ribonucleic acid can be encapsulated in a sustained-release microsphere at a high inclusion rate, and the short chain deoxyribonucleic acid or the short chain ribonucleic acid can be stabilized outside cells and tissues, and their introduction into cells is promoted.

A sustained-release microsphere formulation of the present invention, especially the sustained-release microsphere prepared through a w₁/o/w₂ type emulsion, can protect a short chain deoxyribonucleic acid or a short chain ribonucleic acid against enzymatic degradation, which are otherwise degraded easily by enzymes in blood or tissue, and release stably and persistently the short chain deoxyribonucleic acid or the short chain ribonucleic acid as an active ingredient.

Further according to the present invention, a strong RNAi effect is attained with a quite small amount of a short chain ribonucleic acid.

The sustained-release microsphere of the present invention can release a pharmaceutical nucleic acid for 1 week to 6 months, so that expression of a specific gene can be inhibited not transiently but persistently.

The entire contents of Specification and/or Drawings of Japanese Patent Application No. 2005-254966, from which priority of this application is claimed, are incorporated herein by reference.

### BRIEF DESCRIPTION OF THE DRAWINGS

FIG. 1 illustrates inclusion rates (%) of an antisense oligo-DNA in microspheres prepared by encapsulating a phosphorothioate type antisense oligo-DNA having an inhibition potency against production of an angiogenic inhibition factor VEGF, together with various addition amounts of arginine in a biodegradable, biocompatible polymer (PLGA) (Example 3).

FIG. 2 illustrates inhibition rates of production of VEGF in cells, after transfection of a short chain ribonucleic acid (siRNA) and a phosphorothioate type antisense oligo-DNA, which degrade on a gene level mRNA of an angiogenic inhibition factor VEGF and have an inhibition potency against production of the same, into a mouse-originated cancer cell (S-180) (Example 5): closed circle indicates cases transfected with siRNA, and open circle indicates cases transfected with an antisense oligo-DNA (average value±S.D., n=3).

FIG. 3 illustrates inhibition rates of production of VEGF in S-180 cells transfected with siRNA using as a carrier a positively charged basic substance and a commercially available gene transfection reagent (Example 6).

FIG. 4 illustrates an siRNA release property of a microsphere containing siRNA: open circle indicates a microsphere containing siRNA only, closed circle indicates a microsphere including siRNA together with arginine, and closed triangle indicates a microsphere including siRNA together with PEI (average value±S.D., n=3) (Example 8).

FIG. 5 illustrates a temporal change of tumor volume of tumor bearing mice after administration of siRNA into the tumor at various concentrations: X indicates a control without siRNA administration, closed circle indicates siRNA administration at 1 μM, open circle siRNA at 2 μM, closed triangle siRNA at 5 μM, open triangle siRNA at 10 μM, and open square siRNA at 15 μM (average value±S.E., n=4) (Example 10).

FIG. 6 illustrates a temporal change of tumor volume of tumor bearing mice after administration of a PLGA microsphere containing siRNA into the tumor: open circle indicates administration of PBS only, open triangle a PLGA microsphere without siRNA, closed circle a microsphere containing siRNA only (siRNA dose: 1.3 μg/mouse), closed triangle a microsphere including siRNA together with arginine (siRNA dose: 1.7 μg/mouse), and closed square a microsphere including siRNA together with PEI (siRNA dose: 2.1 μg/mouse) (average value±S.E., n=5) (Example 11).

### BEST MODE FOR CARRYING OUT THE INVENTION

The terms used herein in the present invention have the following meanings.

“Nucleic acid” means a deoxyribonucleic acid (DNA) and/or a ribonucleic acid (RNA).

“A short chain deoxyribonucleic acid or a short chain ribonucleic acid” means an antisense of a short chain DNA or RNA and active derivatives thereof, a ribozyme, and a short double stranded RNA (dsRNA). It means, for example, a ribonucleic acid with 15 to 30 basic pairs (bps), preferably 21 to 29 bp, called as a small interfering RNA (siRNA). An siRNA can be synthesized, can be produced by a cell using DNA or RNA, and may be commercially available. Further an miRNA (micro RNA) having a stem-loop structure is included. An miRNA may be included as a single stranded RNA or as a double stranded RNA with about 70 bases (miRNA precursor). When included as a double stranded RNA with about 70 bases (miRNA precursor), a single stranded RNA is produced by activity of a dicer. Further in “a short chain deoxyribonucleic acid or a short chain ribonucleic acid” are included a nucleic acid aptamer and a decoy nucleic acid. The nucleic acid aptamer is an oligonucleotide (RNA/DNA) with 10 to 85 bases, preferably with 20 to 60 bases, which binds specifically a target protein, penetrates into a pocket of the protein to form a stable 3D structure and has an ability to inhibit the function of the same, wherein it has higher affinity and specificity than an antibody, and inhibits the function differently from an antibody. Examples of aptamers include, but not limited to, aptamers binding various proteins, such as a growth factor (VEGF, PDGF, bFGF), a hormone (neuropeptide Y, LHRH, vasopressin), an enzyme (kinase, protease), a signaling factor, a receptor (neurotensin receptor 1), a membrane protein (PSMA), a transcriptional factor (NF-κB, B2F), and a viral protein. The decoy nucleic acid is a kind of aptamer and can bind a target gene to inhibit expression of the target gene. Examples of decoy nucleic acids include a double stranded type decoy nucleic acid and a ribbon type decoy nucleic acid having higher resistance against a nuclease in serum. More specific examples include decoy nucleic acids recognizing an NF-κB protein, an HIV transcription growth factor (Tat protein), and an NS3 protease of hepatitis C virus. Further included in “a short chain deoxyribonucleic acid or a short chain ribonucleic acid” is a CpG oligo-nucleic acid. The CpG oligo-nucleic acid is an oligo-nucleic acid of about 20 to 30 bases having a CpG motif, usually with a series of cytosine (C) and guanine (G), such as GACGTT, and is capable of activating innate immunity and antigen-specific immunoreaction by co-administration of an antigen. Some sequences have immunosuppressive function. Some single stranded and double stranded RNAs other than those having a CpG motif are known to regulate immunity, and are included in “a short chain deoxyribonucleic acid or a short chain ribonucleic acid”. Such RNA is encapsulated alone or together with an antigen in a microsphere, and used effectively as a single shot vaccine with a strong adjuvant of CpG oligo-nucleic acid, or a single stranded or double stranded RNA, as well as as an immunosuppressive agent or a therapeutic agent treating an autoimmune disease.

“A short chain deoxyribonucleic acid or a short chain ribonucleic acid” according to the present invention includes those which chemical structures are partly modified in order to improve the stability in the body or the affinity. Examples include, but not limited to, introduction of a modified base into a nucleic acid molecule, modification of a phosphate-bonding site, a derivative at the 2′-position of a pentose, introduction of a fluoro-group into a ribose ring, a 4′-thio nucleic acid which is derived by substituting an oxygen atom in a pentose with a sulfur atom.

In the present invention, the length of bases is expressed, in case of a single stranded nucleic acid by a number of bases, and in case of a double stranded nucleic acid by a number of bases or base pairs (bp). In case of a double stranded nucleic acid, the expressions of, for instance, 30 bases and 30 base pairs mean the same length. The length of “a short chain deoxyribonucleic acid or a short chain ribonucleic acid” according to the present invention is 10 to 85 bases, preferably 15 to 60 bases, and more preferably 15 to 30 bases.

siRNA is characterized by being capable of causing RNA interfering (RNAi) to inhibit synthesis of a target protein, thereby only a small amount of siRNA degrades mRNA sequence-specifically in a cell to inhibit expression of the specific gene. The RNAi is one of the target gene knock-down technologies using siRNA, and the use of the same in such versatile research fields is also expected, as search for a new gene which induces a function or differentiation of a cell, determination of an intracellular signaling path, and production of a knock-down cell strain or animal. Further siRNA is expected for a gene therapeutic agent with little side effect, because siRNA can inhibit expression of a gene related to a disease transiently, directly and specifically.

Specific examples of the siRNA include, but not limited to, a short chain nucleic acid capable of inhibiting production of such responsible factors and related factors of various diseases, as: production of a vascular endothelial growth factor and its receptor; production of a Bc1-2 protein presumably involved in canceration of a cell; replication of human immunodeficiency virus (HIV), hepatitis type C and B virus, and other virus causing infectious diseases, such as avian influenza, SARS and West Nile fever; production of a tumor necrosis factor (TNF-α, TNF-β), a monokine, a cytokine, such as an interleukin (IL), a chemokine, a colony-stimulating factor (CSF), and a vascular endothelial growth factor (VEGF), and a receptor thereof involved immune or inflammatory diseases; expression of Fas gene inducing cell apoptosis as one of the causes of liver damage occurred at viral infection or liver transplantation; and production of an apoptosis inhibition factor, such as cFLIP. For example, a tumor can be treated by inhibiting angiogenesis at a tumor site by means of silencing expression of a vascular endothelial growth factor at the tumor site, or can be treated by inducing apoptosis of tumor cells by means of silencing an apoptosis inhibitory factor at the tumor site. By silencing both the expression of the vascular endothelial growth factor and the expression of the apoptosis inhibitory factor at a tumor site, a synergistic effect can be obtained.

“Gene transfer carrier” means a positively charged basic carrier to introduce a nucleic acid, such as a short chain ribonucleic acid (dsRNA, siRNA, etc.), a plasmid and DNA, into a target cell specifically, and the carrier being able to interact electrostatically with siRNA to form a complex.

“A positively charged basic substance” is functionally a gene transfer carrier, and any known as a gene transfer carrier can be used, insofar as it is positively charged and able to interact electrostatically with siRNA to form a complex. Specific examples include positively charged lipid, liposome made thereof, polymer and dendrimer.

A gene transfer carrier, which works as a carrier, is indispensable to introduce a nucleic acid, such as a short chain ribonucleic acid (dsRNA, siRNA, etc.), plasmid and DNA, into a target cell specifically. According to the production method of a particle formulation in the present invention, a negatively charged short chain deoxyribonucleic acid and short chain ribonucleic acid and a positively charged gene carrier interact each other electrostatically, and the short chain deoxyribonucleic acid and the short chain ribonucleic acid is included at high rate in polymeric substances, and the complex of the short chain deoxyribonucleic acid and the short chain ribonucleic acid and the gene carrier is released in the body out of a particle formulation, and the short chain deoxyribonucleic acid or the short chain ribonucleic acid can be effectively introduced into a target cell. There are no restrictions on the gene transfer carrier insofar as it is positively charged and interacts electrostatically with siRNA to form a complex. Examples include positively charged lipid, liposome made thereof, polymer and dendrimer.

More specifically, examples of “positively charged lipid” include dimethyldioctadecylammonium bromide (DDAB), trimethyl-2,3-dioleyloxypropylammonium chloride (DOTMA), N-[1-(2,3-dioleyloxy)propyl]-N,N,N-trimethylammonium chloride (DOTAP), N-2,3-dioleoyloxy-1-propyltrimethylammonium methyl sulfite (DOTAP methosulfate), cholesteryl-3,3-N-dimethyl aminoethyl-carbamate hydrochloride (DS-Chol), 1,2-dimyristyloxypropyl-3-dimethyl-hydroxyethyl ammonium bromide (DMRIE), 2,3-dioleyloxy-N-[2-(sperminecarboxamido)ethyl]-N,N-dimethylammonium trifluoroadetate (DOSPA), O,O′-ditetradecanoyl-N-(α-trimethylammonioacetyl) diethanolamine chloride.

Examples of “a positively charged polymer (cationic polymer)” include polyethylenimine (PEI: linear or branched), and a block copolymer of polyethyleneglycol and poly-L-lysine, and examples of a commercially available gene transfection reagent include Lipofectamine®, Lipofectamine Plus®, jet PEI®, Oligofectamine®, siLentFect®, DMRIE-C®, Transfectin-Lipid®, Effectene®. Polyethylenimine (PEI) includes linear PEI and branched PEI with primary, secondary and tertiary amine, and any of them can be used. There are no restrictions on the molecular weight of PEI. Further, a chemically modified PEI, such as by deacylation, can be used.

Further, other examples can include basic substances as arginine, polyarginine, poly-L-lysine, polyornithine, spermine, protamine and chitosan.

Examples of dendrimer include polyamideamine dendrimer, polyamideamine Starburst dendrimer, dendric polylysine, a cyclodextrin/dendrimer conjugate, Starburst dendrimer.

Additional examples include a cell permeable peptide, such as Tat and a derivative thereof, and a nuclear localization signal, such as NF-κβ

Examples of positively charged materials to be used for production of the particles include arginine, polyethylenimine, poly-L-lysine, poly-L-ornithine, and poly-siLentFect®.

Preferable examples of “a positively charged basic substance” include arginine, especially L(+)-arginine, polyethylenimine, especially a branched type polyethylenimine (PEI), a cell permeable peptide, poly-L-lysine, poly-L-ornithine, and siLentFect®. Poly-L-lysine is preferably constituted of 3 or more lysine residues, more preferably 4 or more lysine residues, especially preferably 10 or more lysine residues. Preferable is a positively charged basic polymeric substance (a cationic polymer), such as polyethylenimine, a cell permeable peptide, poly-L-lysine, poly-L-ornithine, siLentFect®, but not limited thereto.

A plurality of the aforementioned positively charged basic substances may be used in combination.

The positively charged basic substance can associate with a nucleic acid and have a function to include the nucleic acid in a microsphere. Further, with such a polymer as polyethylenimine, a cell permeable peptide, poly-L-lysine, poly-L-ornithine, siLentFect®, higher efficiency of introduction of a microsphere into a cell can be attained.

“An in vivo degradable polymer” used in the present invention means a biodegradable and biocompatible polymer without restrictions insofar as it degrades gradually over a long time period releasing persistently a drug such as siRNA. Examples thereof include, but not limited to, homopolymers, such as aliphatic polymer (polylactic acid, polyglycolic acid, polyhydroxylactic acid, etc.), poly-α-cyanoacrylic acid ester, and polyester, and copolymers of the monomers constituting the above homopolymers.

An especially preferable polymeric substance for the formulation in the present invention is, but not limited to, a polylactic acid/glycolic acid, which is a copolymer of polylactic acid or lactic acid and polyglycolic acid or glycolic acid with the mol ratio of 50/50 to 90/10.

In the present invention “a short chain deoxyribonucleic acid or a short chain ribonucleic acid” and “a positively charged basic substance” are encapsulated in an in vivo degradable polymer, thereby “encapsulation” means a situation wherein the short chain deoxyribonucleic acid or the short chain ribonucleic acid and the positively charged basic substance are contained in a capsule or matrix of the in vivo degradable polymer, as well as a situation wherein the short chain deoxyribonucleic acid or the short chain ribonucleic acid, the positively charged basic substance and the in vivo degradable polymer exist associated with each other and do not break down easily. In the present invention, “encapsulation” may be expressed as “inclusion” or “enclosure”.

In the present invention “a sustained-release microsphere” means a sustained-release particle formulation which has a sustaining function of inhibiting expression of a specific gene by means of regulation of a release or dissolution of a short chain deoxyribonucleic acid or a short chain ribonucleic acid, and is not limited to use for injection or mucosal administration, insofar as it has sustaining release properties. The sustained-release particle formulation may contain known pharmaceutically acceptable additives. Just for convenience, “a microsphere” may be hereunder expressed as “a sustained-release particle formulation”, “microcapsule” or “microparticle”. The microsphere of the present invention can be produced by application of a publicly known method, such as freeze drying, to an emulsion, such as w₁/o/w₂, s/o/w, w/o, o/w and s/w. A preferable emulsion type is w₁/o/w₂ type.

A sustained-release microsphere formulation based on w₁/o/w₂ according to the present invention can be produced using a microcapsule technology, for example using a per se known technique of w₁/o/w₂ drying-in-liquid method, a w₁/o emulsion is prepared by mixing with high speed agitation an internal aqueous phase prepared by dissolving siRNA in the presence of a positively charged basic substance into an oil phase prepared by dissolving an in vivo degradable polymer in an organic solvent; a w₁/o/w₂ emulsion is prepared by adding the w₁/o emulsion into an external aqueous phase with agitation; and the same is dried. Namely an internal aqueous phase, which is prepared by dissolving a water soluble drug, such as a low-molecular-weight compound, a ribonucleic acid and a peptide, preferably a water soluble drug, such as a short chain ribonucleic acid or a short chain deoxyribonucleic acid, and, if required, an drug entrainer, in the presence of a positively charged basic substance into a buffer solution prepared with inorganic materials, such as water and phosphate, or a solution prepared with a surface active polymer, such as polyvinylalcohol, is mixed with high speed agitation with an oil phase, which is prepared by dissolving an in vivo degradable polymer, such as biodegradable and biocompatible polylactic acid/glycolic acid, in an organic solvent, such as dichloromethane, to prepare a w₁/o emulsion, which is then added with agitation into an external water phase, such as an aqueous solution of polyvinylalcohol, which is further agitated to form a w₁/o/w₂ emulsion, out of which an organic solvent, such as dichloromethane, is removed and dried by freeze-drying to form particles encapsulating a drug. The average diameter of particles is several pun to several hundred μm, preferably 10 μm to 150 μm, more preferably 20 μm to 45 μm, especially preferably 20 μm to 30 μm. If the diameter of the particles is smaller than the above range and of the nano-order, they may be phagocytosed by a cell and a nucleic acids in the particles are degraded in a cell, and inclusion of the nucleic acid into the particles is more difficult. If the diameter is larger than the above range, the liquid containing the particles becomes a suspension, which administration by injection becomes difficult. The microspheres of the present invention, when injected subcutaneously, do not enter into blood vessel staying subcutaneously and are able to release gradually the nucleic acid.

The production method of the microsphere is not limited to the above method, but also is carried out by solvent removal in a supercritical fluid or spray drying of a w/o, o/w or s/o emulsion through a w₁/o/w₂ or s/o/w emulsion.

For encapsulation of siRNA, it is recommendable to add gradually an organic solvent, such as hexane, which is compatible with a continuous oil phase, but does not dissolve the in vivo degradable polymer, to the external oil phase through a w/o emulsion and an s/o suspension.

The addition amount of the positively charged basic substance is 1% or more by weight with respect to the internal aqueous phase, preferably 2% or more, further preferably 5% or more, and to maintain a good formulation properties 15% or less, further preferably 10% or less. The water used hereunder is purified water, distilled water, ultrapure water or sterilized water.

For removal of an emulsion solvent, usually the solvent is distilled off at normal temperature under normal pressure with gentle agitation, but a reduced pressure or a gas blow over the surface or inside of the liquid can be also applicable. Further, solvent removal in a supercritical fluid or spray drying can be used. The emulsion type may be s/o/w, w/o, o/w and s/o in addition to w₁/o/w₂.

The sustained-release microsphere including a short chain deoxyribonucleic acid or a short chain ribonucleic acid of the present invention can be as a pharmaceutical composition, namely as a sustained-release microsphere formulation, administered in various forms to a subject.

Therefore the sustained-release microsphere formulation including the short chain deoxyribonucleic acid or the short chain ribonucleic acid of the present invention is useful for treating various diseases including cancer, infectious viral diseases, immunological diseases, inflammatory diseases, intractable diseases, such as liver damage occurred at liver transplantation, diabetic retinopathy, and age-related maculopathy, and lifestyle-related diseases.

Examples of an administration form of the pharmaceutical composition with the microsphere of the present invention include parenteral administration, such as an injectable or implantable formulation, which can be administered intradermally, subcutaneously, intramuscularly, into an eyeball, a joint, an organ tissue and a tumor tissue. The pharmaceutical composition is produced according to a publicly known method, and includes a support, a diluent and an excipient as commonly used in the pharmaceutical field. Examples of a support and an excipient for tablets include gelling agents, lactose, and magnesium stearate. An injectable is prepared by suspending or emulsifying the microsphere in a sterilized aqueous or oily liquid commonly used for an injectable. As an aqueous liquid for an injectable, saline and an isotonic solution including glucose or other adjuvants are used, and polyalcohol, such as polyethyleneglycol, or a nonionic surfactant may be used together. As an oily liquid, sesame oil or soybean oil can be used.

The dose may be determined according to the severity of disease, so that a pharmaceutically effective amount of the composition of the present invention can be administered to a patient. “Administration of a pharmaceutically effective amount” means to administer a patient an appropriate level of a drug required for the treatment. The frequency of administration of the pharmaceutical composition of the present invention is determined appropriately according to the conditions of a patient. A dose is, based on the amount of a short chain deoxyribonucleic acid or a short chain ribonucleic acid included in the microsphere per 1 kg of body weight, 0.0001 to 1000 mg, preferably 0.0001 to 10 mg, more preferably 0.0001 to 0.1 mg. Based on the amount of the microsphere per 1 kg of the body weight, it is 0.1 mg to 100 mg, and preferably 0.2 mg to 50 mg.

When the microsphere including the short chain deoxyribonucleic acid or the short chain ribonucleic acid of the present invention is administered to a subject, the short chain deoxyribonucleic acid or the short chain ribonucleic acid can be released at least for 1 week to 6 months or longer, preferably 1 month to 4 months or longer. Consequently a pharmaceutical composition containing the microsphere of the present invention as an active ingredient may be administered once every 1 week to 6 months, preferably every 1 month to 4 months.

The present invention includes a method for treating various diseases including cancer, infectious viral diseases, immunological diseases, inflammatory diseases, intractable diseases, such as liver damage occurred at liver transplantation, diabetic retinopathy, and age-related maculopathy, and lifestyle-related diseases, by administering the medically effective amount of the sustained-release microsphere of the present invention to a subject requiring a treatment.

The present invention further includes a use of the sustained-release microsphere of the present invention for the production of a pharmaceutical composition for treating various diseases including cancer, infectious viral diseases, immunological diseases, inflammatory diseases, intractable diseases, such as liver damage occurred at liver transplantation, diabetic retinopathy, and age-related maculopathy, and lifestyle-related diseases.

### EXAMPLES

The present invention will now be described in more detail by way of the examples below, provided that the present invention be not limited to the examples.

### Example 1

### A Method for Preparing Microsphere Including an Antisense

The experiment was carried out with an object of establishing a preparation method of a sustained-release microsphere encapsulating in a biodegradable and biocompatible polymer an anti-mouse VEGF antisense oligo-DNA, which inhibits the production of a vascular endothelial growth factor (VEGF) by binding complementarily a messenger RNA (mRNA) relating to the production of VEGF and inhibiting the translation stage in the process of gene expression.

Twenty μL of 2 mM antisense oligo-DNA (21 bases, molecular weight 6360.2, phosphorothioate type) and 0.1 to 10%, based on the liquid quantity of an internal aqueous phase, of L(+)-arginine (Sigma-Aldrich Corp.) were dissolved in 100 μL, of 0.4% polyvinylalcohol solution to form an internal aqueous phase, and 0.5 g of biodegradable, biocompatible polylactic acid-glycolic acid (PLGA; lactic acid/glycolic acid=75/25, Wako Pure Chemical Industries, Ltd.) was dissolved in 2 mL of dichloromethane to form an oil phase. The internal aqueous phase and the oil phase was mixed and subjected to a high speed agitation at 10,000 rpm for 3 minutes to prepare a w₁/o emulsion. The prepared w₁% emulsion was added under agitation to 500 mL of 0.25% polyvinylalcohol solution, and the mixture was agitated at 3,000 rpm for 15 minutes to obtain a w₁/o/w₂ emulsion. The dichloromethane was evaporated off by agitation at 250 rpm for 3 hours, and a supernatant by centrifugation was removed. The residue was washed by distilled water 3 times, and the recovered particles were subjected to freeze-drying to obtain a microsphere including an antisense.

### Example 2

### A Method for Preparing a Sustained-Release Microsphere Including siRNA

The experiment was carried out with an object of establishing a preparation method of a sustained-release fine particles encapsulating in PLGA a short chain ribonucleic acid siRNA which can inhibit the synthesis of VEGF by degrading mRNA related to the production of VEGF.

Twenty-five μl of 350 nM concentration anti-mouse VEGF siRNA (21 bp, molecular weight 13345.4) and 7.5 μg of L(+)-arginine or 5 μg of branched type polyethylenimine (PEI, molecular weight 25 kDa, Sigma-Aldrich Corp.) were dissolved in 100 μL of 0.4% polyvinylalcohol solution to form an internal aqueous phase. In 3 mL of dichloromethane 0.5 g of the PLGA used in Example 1 was dissolved to form an oil phase. The internal aqueous phase and the oil phase were mixed and subjected to a high speed agitation at 10,000 rpm for 2 minutes to prepare a w₁/o emulsion. The prepared w₁/o emulsion was then added under agitation to 500 mL of 0.25% polyvinylalcohol solution, and the mixture was agitated at 3,000 rpm for 3 minutes to obtain a w₁/o/w₂ emulsion. The dichloromethane was evaporated off by agitation at 250 rpm for 3 hours, and a supernatant by centrifugation was removed. The residue was washed by distilled water 3 times, and the recovered particles were subjected to freeze-drying to obtain a microsphere including siRNA.

### Example 3

### Inclusion Rate (%) of an Antisense Oligo-DNA

The microsphere including an antisense oligo-DNA prepared in Example 1 was observed under a microscope, and further with a photomicrograph the Feret horizontal diameter was measured to calculate the average particle size. Further, 25 mg of the microsphere was placed in a test tube, to which 0.5 mL of acetonitrile was added to dissolve the PLGA component and 0.5 mL of a phosphate-buffer solution (pH 6.0) was added. The mixture was shaken for 2 hours, and centrifuged at 5,000 rpm for 20 minutes. The supernatant was analyzed by HPLC to determine the quantity of the antisense oligo-DNA encapsulated in the microsphere. The inclusion rate (%) of the antisense oligo-DNA in the microsphere was calculated as the ratio of a measured quantity of the antisense oligo-DNA to the total mass (defined as 100%) of the formulated quantities of the solid components used at the preparation of the particles. The analysis conditions of HPLC were as shown below.

### Apparatus:

Shimadzu HPLC system (SCL-10Avp system controller, LC10ADvp pump, DGU-12A degasser, SPD-10Avp UV detector, SIL-10Avp auto-injector, CTO-10ASvp column oven, C-R8A printer)

### Column:

TSKgel Oligo DNA RP, 4.6 mm×15 cm, TOSOH
   Mobile phase:
A: 0.1 M triethylamine acid (TEAA)
B: acetonitrile
A/B (90/10) to A/B (70/30)
linear gradient (45 minutes)

Flow rate: 1 mL/min

Detection: UV (260 nm)

Injection quantity: 10 μL

### (Result)

Spherical particles of the prepared microsphere including an antisense oligo-DNA were observed under a microscope, and confirmed that the average particle sizes were all in the range of 30 to 45 μm, which particle sizes were easily passable through a injection needle and appropriate for an injectable.

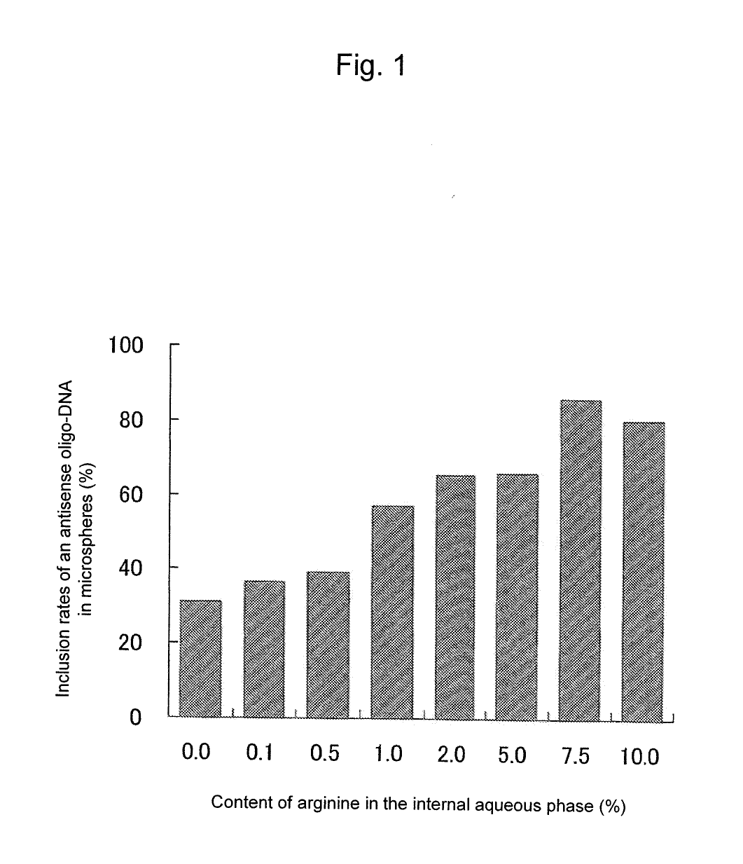

As illustrated in FIG. 1, the inclusion rate of the antisense oligo-DNA in a microsphere varies with the ratio of arginine added at the preparation of the particles in the internal aqueous phase, and the inclusion rate increases with the increase of the content of arginine added. Especially, the inclusion rate reached approximately 80%, if arginine was added 7.5 weight % or more of the internal aqueous phase, which indicated that by adding an appropriate amount of a positively charged basic substance, such as arginine, a microsphere including an antisense oligo-DNA can be prepared at a high inclusion rate.

### Example 4

### Evaluation of Release Properties of an Antisense DNA Out of a Microsphere, Using a Residual Rate as an Index

Twenty-five mg of the microsphere prepared in Example 1 was weighed and placed into a test tube with a stopper, to which 1.5 mL of 0.1 M phosphate buffer (pH 7.4) at 37° C. was added. The mixture was subjected to a release test for 28 days at 37° C. with a stirrer. After elapse of a defined time period, the mixture was centrifuged at 5,000 rpm for 20 minutes, the supernatant was removed, and the obtained precipitate (the microsphere) was mixed with 0.5 mL of acetonitrile to dissolve the PLGA component. To the mixture 0.5 mL of a phosphate buffer (pH 6.0) was added and mixed vigorously, after shaking for 2 hours the mixture was centrifuged at 5,000 rpm for 20 minutes. The supernatant was analyzed by HPLC to determine the quantity of the antisense DNA remained in the microsphere. The residual rates (%) were calculated as the ratios of the quantity of the antisense DNA remained in the microsphere at various time points to the quantity of the antisense DNA in the microsphere before the test, which was defined as 100%. The release properties of an antisense DNA out of a microsphere were evaluated using the residual rate as an index.

The analysis conditions of HPLC are same as in Example 3.

### (Result)

It was demonstrated that the microsphere prepared by adding 5% or more of arginine to the internal aqueous phase released persistently and stably the antisense DNA for 2 months.

### Example 5

### Inhibition Rate (%) of Production of VEGF

Culture cells suspended in DMEM medium with serum: Cancer cells originated from murine kidney Sarcoma 180 (S-180) were seeded on a 24-well culture plate at the density of 1×10⁵ cells/well, and precultured under the conditions at 37° C., 5% CO₂. After 24 hours, the cells were washed by a phosphate buffered saline (PBS) and the medium was changed to a serum-free medium RPMI1640, and 0.13 μg of the siRNA used in Example 2, or 3.25 μg of the antisense oligo-DNA used in Example 1 was added to each well of the culture plate, which was then subjected to transfection under the conditions at 37° C., 5% CO₂ for 12 hours. Then the cells were washed by PBS and a serum-free medium RPMI1640 was added and left stand under the conditions at 37° C., 5% CO₂, and the quantity of VEGF in the medium was measured by an enzyme-linked immunoassay (ELISA) at 12 hour-intervals during 72 hours. The inhibition rate (%) of the production of VEGF was calculated as a ratio of the VEGF quantity in the sample medium per unit cell to the VEGF quantity per unit cell in the medium with the cells only, which is defined as 100%.

### (Result)

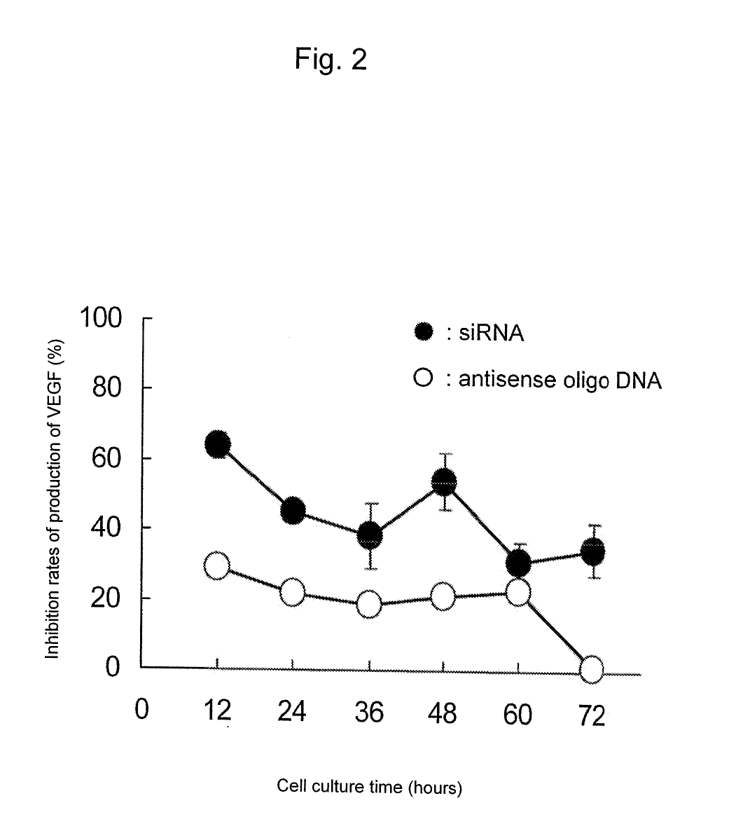

As shown in FIG. 2, siRNA showed a higher inhibition rate of the production of VEGF than the antisense oligo-DNA. The dose of siRNA was 1/25 of that of the antisense oligo-DNA, which indicated that with an extremely small amount of a short chain ribonucleic acid a high RNAi effect can be obtained. The effect of the antisense DNA disappeared after 3 days and this short effective period is a problem. According to the test with siRNA the inhibition lasted 3 days, but thereafter the inhibition rate decreased gradually. It is believed that the persistence of effect is usually up to about 1 week. The test indicated the necessity of a sustained-release formulation of a short chain ribonucleic acid for persistent functioning.

### Example 6

### Inhibition Rate (%) of Production of VEGF

The object of the test was to assess the necessity of a gene carrier by evaluating the RNAi effect in the case of introducing siRNA, having the inhibition effect on the production of VEGF, into cells, together with a basic substance or a commercially available transfection reagent.

As gene transfer carriers were used L(+)-arginine (7.5 μg), branched type polyethylenimine (PEI, Mw 2.5 kDa, 0.1 μg), jetPEI (0.8 mL, N/P ratio=2), Lipofectamine (2 μg), SiLentfect (1.6 μg), and 0.13 μg of the siRNA used in Example 2 was mixed with the respective substances to form complexes. As in Example 5, S-180 cells suspended in DMEM medium with serum were seeded on a 24-well culture plate at the density of 1×10⁵ cells/well, and precultured under the conditions at 37° C., 5% CO₂. After 24 hours, the cells were washed by a phosphate buffered saline (PBS) and the medium was changed to a serum-free medium RPMI1640, and 0.13 μg of siRNA alone, or the complex of siRNA and the carrier prepared above was added to each well of the culture plate, which was then subjected to transfection under the conditions at 37° C., 5% CO₂. After 12 hours the cells were washed by PBS and a serum-free medium RPMI1640 was added and left stand under the conditions at 37° C., 5% CO₂. After 12 hours the quantity of VEGF in the medium was measured by ELISA, and the inhibition rate (%) of the production of VEGF was calculated as in Example 5.

### (Result)

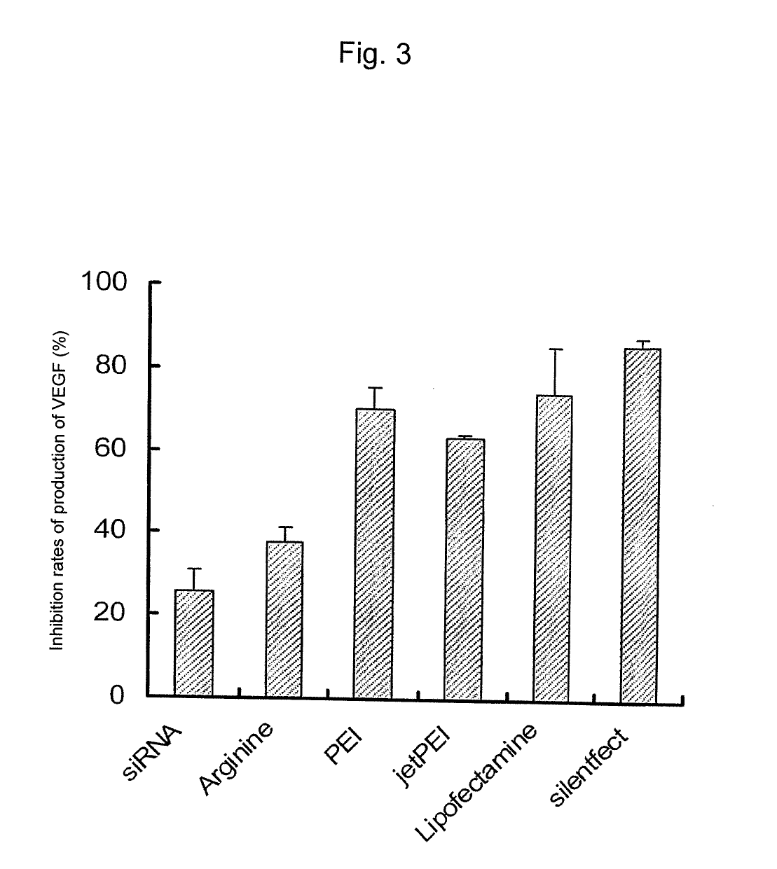

As shown in FIG. 3, it was clearly demonstrated that by administering a complex formed by electrostatic interaction between a positively charged gene carrier and negatively charged siRNA, the inhibition rate (%) of the production of VEGF become remarkably higher than single administration of siRNA. The result indicates that a gene carrier is necessary to deliver siRNA into a cell and to induce a high RNAi effect.

### Example 7

### Inclusion Rate (%) of siRNA in a Microsphere

The microsphere prepared in Example 2 was observed under a microscope, and further with a photomicrograph the Feret horizontal diameter was measured to calculate the average particle size. Further, 25 mg of the microsphere was placed in a test tube, to which 0.5 mL of acetonitrile was added to dissolve a PLGA component and 0.5 mL of phosphate-buffer solution (pH 6.0) was added. The mixture was shaken for 2 hours, and centrifuged at 5,000 rpm for 2 minutes. The supernatant was analyzed by HPLC to determine the quantity of the siRNA encapsulated in the microsphere. The inclusion rate (%) of siRNA in the microsphere was calculated as the ratio of a measured quantity of siRNA to the total mass (defined as 100%) of the formulated quantities of the solid components used at the preparation of the particles.

The analysis conditions of HPLC are same as Example 3.

### (Result)

It was confirmed by observation under a microscope that any of the prepared microspheres prepared in Example 2 encapsulating siRNA alone, encapsulating siRNA and arginine, and encapsulating siRNA and PEI are spherical particles. Further, as shown in Table 1, the average particle sizes of the microspheres were all in the range of 30 to 45 μm, which particle sizes were easily passable through a injection needle, and therefore it was confirmed that their sizes were appropriate for an injectable. The inclusion rate of siRNA in the microsphere encapsulating siRNA alone was about 48%. In contrast thereto, if a positively charged basic substance, arginine, was added, the rate was as high as about 64%, and if PEI was added the inclusion rate reached a high value of about 80%. From the above results, it was demonstrated that, in order to include siRNA in a microsphere at a high inclusion rate, addition of siRNA together with a positively charged substance is effective, and especially with PEI, which is used also as a gene transfection reagent, the inclusion efficiency becomes higher.

TABLE 1

| | | Inclusion |
|---|---|---|
| Basic substance added to | Average particle size of | rate of siRNA |
| internal aqueous phase | particles (μm) | into a particle (%) |
| None | 44.5 ± 22.1 | 48.62 ± 0.14 |
| L(+)-arginine | 34.8 ± 16.8 | 64.32 ± 3.74 |
| Polyethylenimine | 37.2 ± 21.6 | 80.26 ± 6.92 |

### Example 8

### Release Behavior of siRNA out of a Microsphere

The test was carried out to study the release behavior of siRNA out of a microsphere encapsulating siRNA in PLGA.

Twenty-five mg of the microsphere prepared in Example 2 was weighed and placed into a test tube with a stopper, to which 1.5 mL of 0.1 M phosphate buffer (pH 7.4) at 37° C. The mixture was subjected to a release test for 28 days at 37° C. with a stirrer. After elapse of a defined time period, the mixture was centrifuged at 5,000 rpm for 20 minutes, the supernatant was removed, and the obtained precipitate was mixed with 0.5 mL of acetonitrile to dissolve the PLGA component. To the mixture 0.5 mL of phosphate buffer (pH 6.0) was added and after shaking for 2 hours the mixture was centrifuged at 5,000 rpm for 2 minutes. The supernatant was analyzed by HPLC to determine the quantity of siRNA remained in the microsphere. The residual rates (%) were calculated as the ratios of the quantity of siRNA remained in the microsphere at various time points to the quantity of siRNA in the microsphere before the test, which was defined as 100%. The release properties of siRNA out of a microsphere were evaluated using the residual rate as an index.

The analysis conditions of HPLC are same as in Example 3.

### (Result)

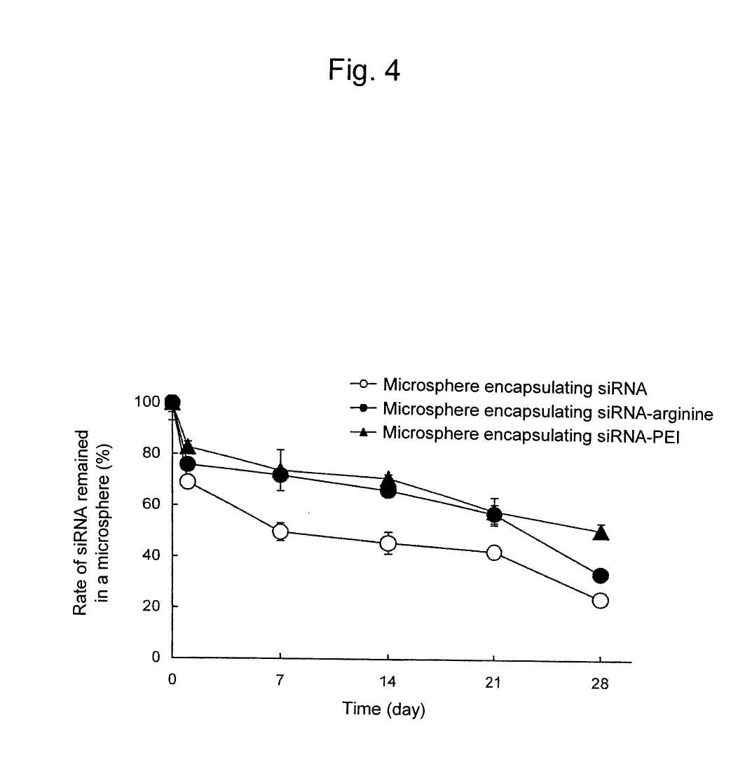

As shown in FIG. 4, it was recognized that the initial burst of the microsphere added with arginine or PEI was lower than the microsphere encapsulating siRNA only, and the siRNA was persistently released for 28 days. It was therefore demonstrated that by adding a positively charged basic substance, such as arginine or PEI, into the internal aqueous phase during the preparation stage of the microsphere, the inclusion rate as well as the initial burst can be improved and the control of the release rate is possible.

### Example 9

### Evaluation of the Inhibition Rate (%) of Production of VEGF

As demonstrated by Example 7, the microsphere prepared in Example 2 showed persistent release properties in the evaluation of the in vitro release property test using a buffer solution. Consequently, a test was carried out to evaluate the inhibition effect on the production of VEGF by the microsphere including siRNA similar to Examples 5 and 6 by means of an experiment system using cells.

As in Example 5, S-180 cells suspended in a DMEM medium were seeded on a 24-well culture plate at the density of 1×10⁵ cells/well, and precultured under the conditions at 37° C., 5% CO₂ for 24 hours. Then the cells were washed by a phosphate buffered saline (PBS) and the medium was changed to a serum-free medium RPMI1640, and meshed chambers containing respectively 10 mg of a microsphere with only PLGA prepared in Example 2, a microsphere with only siRNA, and a microsphere with arginine and siRNA were placed on the cells in the respective wells and left stand under the conditions at 37° C., 5% CO₂. After 12 hours the medium samples were taken and the quantity of VEGF in the medium was measured by ELISA. The inhibition rate (%) of the production of VEGF was calculated as a ratio of the VEGF quantity per unit cell in the medium used for the microsphere with siRNA, or the microsphere with siRNA and arginine to the VEGF quantity per unit cell in the medium used for the microsphere with only PLGA, which is defined as 100%. Since a serum-free medium was used, the cell could not be viable for a long period. Consequently, at intervals of 48 hours the chambers with the microspheres were replaced on fresh cells precultured separately, and the VEGF quantity in the medium was measured as above after 12 hours. The procedure was repeated for 17 days to evaluate the 17-day RNAi effect of siRNA persistently released out of the microsphere.

### (Result)

As shown by the inhibition rates of the production of VEGF in Table 2, for 12 hours after the start of the test there was shown no significant difference of the inhibition effects on the production of VEGF between the microsphere encapsulating siRNA only, and that encapsulating siRNA and arginine. But it was recognized that with the microsphere encapsulating siRNA only, the inhibition rate of the production of VEGF after 12 hours decreased over time, and no persistent RNAi effect by siRNA was obtained. However, it was demonstrated that with the siRNA microsphere encapsulating arginine together, a remarkable RNAi effect persisted until day 16.5 in contrast to the microsphere encapsulating siRNA only.

TABLE 2

| | Microsphere | SiRNA microsphere |
|---|---|---|
| | encapsulating siRNA | encapsulating siRNA |
| Time (day) | only | and arginine together |
| 0.5 | 74.8 ± 5.5 | 70.8 ± 4.4 |
| 2.5 | 41.3 ± 13.9 | 56.4 ± 1.6 |
| 4.5 | 48.6 ± 5.7 | 49.0 ± 2.9 |
| 6.5 | 12.2 ± 0.9 | 51.6 ± 3.6 |
| 8.5 | 26.1 ± 9.3 | 60.9 ± 21.0 |
| 10.5 | 22.0 ± 18.7 | 62.2 ± 12.1 |
| 12.5 | 1.6 ± 14.7 | 56.6 ± 7.1 |
| 14.5 | 3.8 ± 7.4 | 41.2 ± 8.2 |
| 16.5 | 22.6 ± 7.3 | 60.3 ± 0.8 |

### Example 10

### Evaluation of the siRNA Effect In Vivo by a Change of Tumor Volume as an Index

A test was carried out to evaluate the effect of siRNA in vivo by administering siRNA with various concentrations to tumor bearing mice, and using a change of tumor volume as an index.

Production of tumor bearing mice: As in Example 5 S-180 cells were precultured in DMEM medium with serum under the conditions at 37° C., 5% CO₂. The cells were washed by PBS and suspended in a serum-free medium RPMI1640. The S-180 cells (5×10⁶ cells/300 μL) were injection-transplanted subcutaneously to the back of 8-week-old female ICR mice. On day 6 after transplantation, when the tumor volume reached 50 mm³ or more, the mice were judged as tumor bearing and used for the test.

Into the tumors of the tumor bearing mice on day 6 after the transplantation of S-180 by the method described above, the siRNA used in Example 2 was administered at various concentrations of 1, 2, 5, 10 and 15 μM. On day 1, 3, 5, 7, 10 and 14 thereof the major axis and minor axis of the tumor were measured, and the tumor volume was calculated using the following formula.

tumor volume (mm³)=(tumor minor axis)²×tumor major axis/2

### (Result)

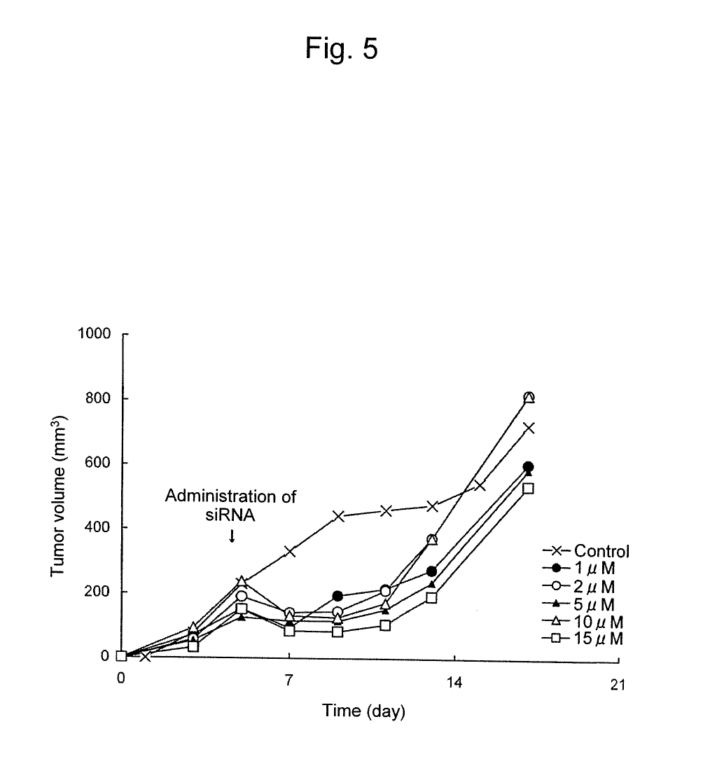

As shown in FIG. 5, in case of the control without the administration of siRNA, the tumor volume increased over time, while in the mice administered the siRNA solution into the tumors, the tumor growth was remarkably inhibited at any administered concentration. There was shown a tendency that the inhibition of the tumor growth was dependent on the siRNA concentration. However, after day 7 of the siRNA administration, there was shown a tendency that the tumor volume increased rapidly, clearly indicating that the persistent RNAi effect can not be obtained by a single administration of siRNA alone irrespective of the concentrations.

### Example 11

### Evaluation of the RNAi Effect In Vivo Using a Microsphere Including siRNA

According to Example 9, with a single administration of siRNA alone to a tumor bearing mouse, the remarkable RNAi effect was recognized. However the effect was transient and the longest persistence period of effect was about 7 days. Consequently, another test was carried out to evaluate the RNAi effect in vivo using the microsphere including siRNA prepared in Example 2.

Tumor bearing mice were produced as in Example 9, and the following test was carried out on day 6 after transplantation, when the tumor volume reached 50 mm³ or more.

Although 25 μL of 350 nM siRNA was added to the internal aqueous phase in Example 2, in this example 20 μL of the same was changed to 25 μL, a microsphere including siRNA was prepared according to the w₁/o/w₂ drying-in-liquid technique shown in Example 2.

A microsphere with PBS only and without PBS and siRNA was administered into a tumor of a tumor-bearing mouse as the control. A PBS solution suspending 10 mg of a microsphere including siRNA was administered into a tumor of a tumor-bearing mouse. The tumor volume was measured at 2-day intervals after the administration by the method similar to Example 7.

### (Result)

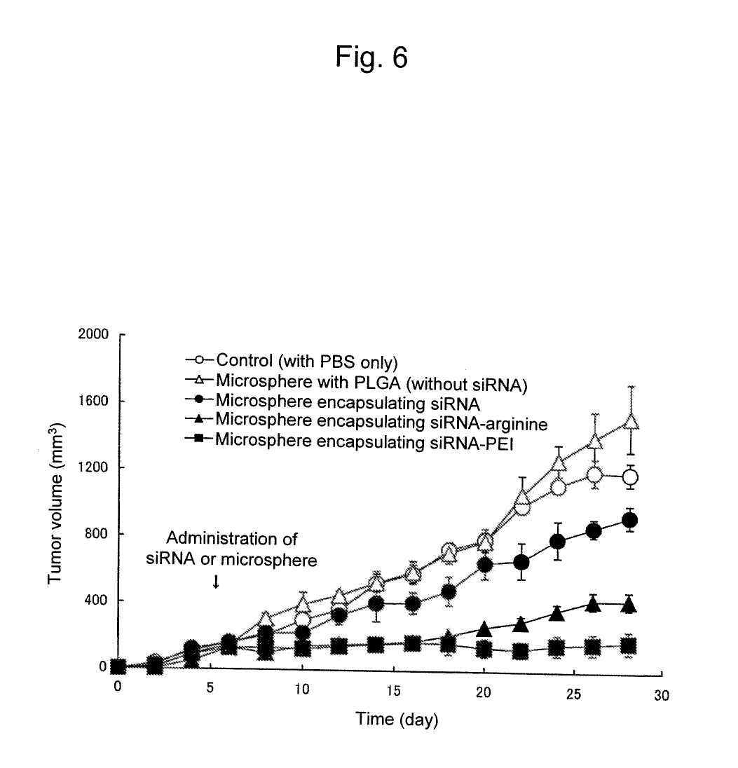

As shown in FIG. 6, increase of the tumor volume was rapid in case of the control, while the inhibition of the tumor growth by the microsphere including siRNA was recognized. It became clear that the inhibition due to the RNAi effect by the microsphere including siRNA was, compared to the microsphere including siRNA only, more remarkable with the siRNA microsphere including siRNA and arginine or PEI together, which persisted as long as about 1 month. The above has indicated that a sustained release microsphere can be prepared, which can release stably and persistently siRNA for long period to obtain in vivo a persistent RNAi effect, by forming a microsphere by means of encapsulating siRNA in an in vivo degradable polymer using a positively charged basic substance as a carrier.

### Example 12

### Preparation of a Sustained Release Microsphere Including Anti-cFLIP siRNA

A sustained release fine particles encapsulating, in PLGA, both a short chain ribonucleic acid siRNA to inhibit the synthesis of a cellular FLICE-inhibitory protein (cFLIP), which is an inhibiting factor of apoptosis, by degrading mRNA related to production of cFLIP, and siRNA to inhibit the production of VEGF, were prepared.

An internal aqueous phase was formed by dissolving 25 μL of 40 μM concentration anti-mouse cFLIP (23 bp, molecular weight 14544), 25 μL of 40 μM concentration anti-mouse VEGF (21 bp, molecular weight 13345.4), and 500 μg of branched type polyethylenimine (PEI, molecular weight 25 kDa, Sigma-Aldrich Corp.) in 100 μL of 0.4% polyvinylalcohol solution. An oil phase was formed by dissolving 0.5 g of PLGA used in Example 1 in 3 mL of dichloromethane. The mixture of the internal aqueous phase and the oil phase was subjected to high speed agitation at 10,000 rpm for 2 minutes to prepare a w₁/o emulsion. The prepared wₕ% emulsion was then added into 500 mL of 0.25% polyvinylalcohol solution with agitation, the mixture was agitated at 3,000 rpm for 3 minutes to obtain a w₁/o/w₂ emulsion. The emulsion was further agitated at 250 rpm for 3 hours to evaporate off dichloromethane, and centrifuged to remove the supernatant. After washing with distilled water 3 times, the recovered particles were subjected to freeze-drying to obtain a microsphere including siRNA.

The average particle size of the obtained microsphere was about 23 μm, and the content of siRNA was about 83%.

The publications, patents and patent applications referred to herein are hereby incorporated herein by reference.

### INDUSTRIAL APPLICABILITY

The sustained-release microsphere according to the present invention, especially a w₁/o/w₂ type sustained-release microsphere, can encapsulate in the sustained-release microsphere a larger amount of siRNA (small interfering RNA) than conventional ones, and release a drug over the long time period.

Consequently its use as a gene formulation for gene therapy is very promising.

1. A sustained-release microsphere comprising a short chain deoxyribonucleic acid or a short chain ribonucleic acid as an active ingredient and 1 weight % to 10 weight % of a positively charged basic substance which can form a complex with said nucleic acid by means of electrostatic interaction.

2. The sustained-release microsphere according to claim 1, wherein the short chain deoxyribonucleic acid or the short chain ribonucleic acid has a single strand or double strand structure, and the length of 10 to 85 bases.

3. The sustained-release microsphere according to claim 1, wherein the short chain deoxyribonucleic acid or the short chain ribonucleic acid has a single strand or double strand structure, and the length of 15 to 30 bases.

4. The sustained-release microsphere according to claim 1, wherein the short chain ribonucleic acid is siRNA with the length of 15 to 30 bases.

5. The sustained-release microsphere according to claim 1, wherein the positively charged basic substance is a cationic polymer.

6. The sustained-release microsphere according to claim 1, wherein the positively charged basic substance is selected from the group consisting of arginine, polyethylenimine (PEI), a cell permeable peptide, poly-L-lysine, poly-L-ornithine, and siLentFect®.

7. The sustained-release microsphere according to claim 6, wherein the positively charged basic substance is selected from the group consisting of polyethylenimine (PEI), a cell permeable peptide, poly-L-lysine, poly-L-ornithine, and siLentFect®.

8. The sustained-release microsphere according to claim 1, which further comprises an in vivo degradable polymer.

9. The sustained-release microsphere according to claim 8, wherein the in vivo degradable polymer is a copolymer of polylactic acid and polyglycolic acid or a copolymer of lactic acid and glycolic acid.

10. The sustained-release microsphere according to claim 1, wherein the short chain deoxyribonucleic acid or the short chain ribonucleic acid as an active ingredient can be injected intradermally, subcutaneously, intramuscularly, into an eyeball, a joint, an organ tissue or a tumor tissue.

11. A pharmaceutical composition comprising the sustained-release microsphere according to any one of claims 1 to 10 as an active ingredient.

12. An anticancer agent comprising the sustained-release microsphere according to claim 1 as an active ingredient, wherein the short chain deoxyribonucleic acid or the short chain ribonucleic acid can inhibit growth of tumor cells.

13. A method, based on a w₁/o/w₂ emulsion drying-in-liquid technique, for producing the sustained-release microsphere according to claim 1, characterized in that the method comprises the steps of:
forming a w₁/o emulsion by mixing with high speed agitation an internal aqueous phase prepared by dissolving siRNA in the presence of a positively charged basic substance, into an oil phase prepared by dissolving an in vivo degradable polymer in an organic solvent;
forming a w₁/o/w₂ emulsion by adding the w₁/o emulsion into an external aqueous phase solution with agitation; and
drying the same.

14. A method for producing the sustained-release microsphere according to claim 1, characterized in that a w/o, o/w or s/o emulsion through a w₁/o/w₂ or s/o/w emulsion, is subjected to solvent removal in a supercritical fluid or spray drying.

15. The production method according to claim 14, characterized in that an organic solvent having compatibility with a continuous oil phase, but not solubility of an in vivo degradable polymer, is gradually added to an external oil phase through a w/o emulsion or an s/o suspension to have the short chain deoxyribonucleic acid or the short chain ribonucleic acid encapsulated.

16. The production method according to claim 15, wherein the in vivo degradable polymer is a copolymer of polylactic acid and polyglycolic acid or a copolymer of lactic acid and glycolic acid.
